(19) United States
(12) Patent Application Publication
(10) Pub. No.: US 2010/0151012 A1
(43) Pub. Date: Jun. 17, 2010

(54) STABILISED PHARMACEUTICAL COMPOSITION CONTAINING PREGABALINE

(75) Inventors: Julia Schulze-Nahrup, Neuried (DE); Alexander Lehmann, Zurchel (DE); Frank Muskulus, Grobenzell (DE)

Correspondence Address: HEDMAN & COSTIGAN P.C., 1185 AVENUE OF THE AMERICAS, NEW YORK, NY, 10036, US

(73) Assignee: RATIOPHARM GMBH, Ulm (DE)

(21) Appl. No.: 12/450,858

(22) PCT Filed: Apr. 23, 2008
(86) PCT No.: PCT/EP2008/003285

§ 371 Date: Oct. 15, 2009

(30) Foreign Application Priority Data
Apr. 23, 2007 (DE) 10 2007 019 071.0

(51) Int. Cl. A61K 9/48; A61K 31/195; A61K 9/14; A61P 25/08; A61K 9/20

(52) U.S. Cl. 424/452; 514/561; 424/489; 424/465

(57) ABSTRACT

Solid pharmaceutical composition containing: (a) pregabaline as active principle and (b) one or several pharmaceutical auxiliary agents, the composition being essentially free from saccharides and comprising no further amino acids, apart from pregabaline.

The invention relates to a solid pharmaceutical composition, in particular a stabilised solid pharmaceutical composition containing

(a) pregabaline as active principle and

(b) one or several pharmaceutical auxiliary substances,

the composition being essentially free from saccharides and comprising no further amino acids, apart from pregabaline.

“Pregabaline” is the INN designation for 4-amino-3-(2-methyl propyl) butyric acid. The compound has the following structure:

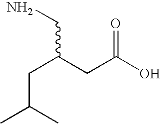

In English, pregabaline is usually referred to as “3-(aminomethyl)-5-methylhexanoic acid”. Pregabaline is a pharmaceutical from the group of anticonvulsives. Pregabaline has been approved for the treatment of epilepsy and of neuropathic pain.

Within the context of this patent application, the expression “pregabaline” comprises the S-isomer, the R-isomer and an R/S-isomer mixture. In a preferred embodiment, “pregabaline” consists of S-pregabaline:

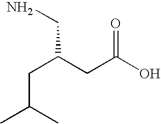

Various pregabaline formulations are known in the state of the art.

EP 641 330 A describes the preparation of pregabaline and mentions pharmaceutical compositions only in general terms.

EP 1 377 318 A2 (WO 02/078747) describes the formation of lactose conjugates as decomposition product during the formulation of capsules containing pregabaline as active principle and lactose as auxiliary substance. However, in view of the requirements for approval or the product quality, such conjugates are undesirable. According to EP 1 377 318, the conjugates are formed both by Maillard reaction with lactose and by Maillard reaction with the lactose building blocks of galactose and glucose. When using saccharides such as e.g. cellulose or derivatised saccharides such as e.g. micro-crystalline cellulose as auxiliary substances, the Maillard reaction can also lead to the formation of conjugates.

EP 1 194 125 A (WO 01/03672) relates to the preparation of taste-masked immediate-release granules useful for making rapid-release pharmaceutical tablets and comprising pregabaline and a polysaccharide, i.e. ethylcellulose.

EP 1 077 692 (WO 99/59573) describes the use of alpha-amino acids for stabilising pregabaline. In particular, a pregabaline formulation is disclosed which comprises magnesium stearate, talcum and L-leucine as auxiliary substances.

EP 1 077 691 A1 (WO 99/59572) relates to pregabaline formulations with a humectant such as e.g. polypropylene glycol as essential component.

WO 2006/121557 describes pregabaline which is essentially free from lactams. A pregabaline formulation comprising starch and microcrystalline cellulose is proposed.

WO 2006/078811 relates in particular to compositions of gabapentin. The compositions specifically disclosed comprise polysaccharides.

WO 2007/079195 relates to retard formulations of gabapentin or pregabaline. Specific examples relate to gabapentin.

WO 2007/052125 describes specific formulations comprising matrix forming agents (polyvinyl acetate or PVP) and a swelling agent (CR-PVP).

WO 2007/053904 relates to a multi-step process for the control of particle size but is not specific for pregabaline.

EP 1 395 242 A (WO 02/094220) relates to liquid preparations containing short-chain polyhydric alcohols. Solid components are considered as part of a two-component system. Specific examples relate to gabapentin.

EP 1 543 831 A (WO 2005/063229) relates to an aqueous pregabaline preparation with a stabilized pH range. WO 2006/008640 relates to a non-aqueous suspension containing a drug having an unpleasant taste. WO 2007/107835 relates to liquid stabilized preparations containing a C2-C6 polyhydric alcohol.

WO 01/24791 relates to liquid and solid formulations comprising a NK1-receptor antagonist and a GABA-analogue for the treatment of psychiatric disorders, i.e. to synergistic compositions comprising two active ingredients. The solid formulation comprises corn starch as an ingredient.

EP 1 100 467 (WO 00/07568) relates to a method for making coated gabapentine or pregabaline particles.

WO 2003/068186 relates to pharmaceutical formulations for improved absorption and multistage release of active agents. Specific examples relate to formulations comprising other active ingredients than pregabaline.

WO 2005/051384 relates to the stabilisation of amino acid compositions with calcium carbonate. Specific examples relate to compositions comprising a polysaccharide.

WO 2006/108151 describes different crystalline forms of pregabaline, and in particular different polymorphic forms of pregabaline.

The auxiliary substances used in the state of the art can, however, trigger undesirable negative reactions in patients. In the case of the saccharides used as auxiliary substances, intolerances may arise in particular with lactose. Cases of lactose intolerance are the most wide-spread cases of food intolerance world-wide. This may lead to restrictions of use. However, it has become apparent from the state of the art that the use of lactose or of amino acids is desirable for reasons of stability.

Particularly desirable would be compositions which are stable insofar as they do not comprise decomposition products or derivatives of the active ingredient, of which the dissolution kinetics and bioavailability remains stable after storage, and in particular compositions wherein initially present polymorph-forms of the active ingredient are not subject to substantial changes.

A further object consisted of providing a pharmaceutical composition which exhibits an advantageous stability under the following storage conditions and, in particular, has fewer decomposition products than the formulations known from the state of the art:

1) 60° C. for 4 weeks (stress stability test);

2) 40° C. at a relative atmospheric humidity of 75% for 12 weeks; or

3) 30° C. at a relative atmospheric humidity of 65% for 12 weeks; or

4) 25° C. at a relative atmospheric humidity of 60% for 12 weeks.

Moreover, it was the object of the invention to provide a formulation which is highly suitable for delayed release forms of presentation, in particular for delayed release tablets

It has now unexpectedly been found that an advantageous pregabaline formulation can be provided which, on the one hand, is largely free from saccharides such as lactose and, on the other hand, requires no amino acids (apart from pregabaline as active principle) for stabilisation. It has proved to be particularly unexpected that the pregabaline formulations according to the invention have advantageous properties with a view to stability (and above all stress stability).

The subject matter of the invention is consequently a solid pharmaceutical composition containing

(a) pregabaline as active principle and

(b) one or several pharmaceutical auxiliary agents

the composition being essentially free from saccharides and comprising no further amino acids, apart from pregabaline.

Compositions according to the present invention are substantially stable insofar as no polymorphic change of pregabaline anhydrate into one of two forms as described in WO 2006/108151 A1 could be observed.

The composition according to the invention is essentially free from saccharides. This should be understood to mean that, in general, it contains less than 10% by weight, preferably less than 5% by weight, more preferably less than 2% by weight and in particular less than 0.5% by weight of saccharides, based on the total weight of the pharmaceutical composition.

The composition according to the invention comprises, apart from pregabaline, essentially no further amino acids. This should be understood to mean that component (b) in general contains less than 5% by weight, preferably less than 2% by weight and more preferably less than 0.5% by weight and in particular less than 0.01% by weight of amino acids, based on the total weight of the pharmaceutical composition.

Preferably, the composition comprises no other active ingredient than pregabaline.

The term “saccharide” should commonly be understood to mean sugar with a hydroxyaldehyde or hydroxyketone structure. The term “saccharide” comprises in general monosaccharides, disaccharides and polysaccharides. The term “monosaccharide” comprises the pentoses arabinose, ribose, xylose and the hexoses glucose, mannose, galactose and fructose. The term “disaccharide” comprises sucrose, trehalose, lactose and maltose. The term “polysaccharide” comprises starch, glycogen and cellulose. Moreover, the term “polysaccharide” also comprises cellulose ethers such as e.g. ethyl cellulose, carboxymethylcellulose, hydroxypropylcellulose (HPMC) or hydroxypropylcellulose. The term saccharide includes also molecules or compound with one or several glucose monomers contained therein.

However, the term “saccharide” does not comprise reduction products of the hydroxyaldehydes or hydroxyketone such as e.g. hexites (hexahydric alcohols) or pentites (pentahydric alcohols).

In a possible embodiment, the composition according to the invention comprises pregabaline with a mean particle size of less than 250 μm, more preferably of 0.1 to 200 μm, in particular more than 10 μm to 150 μm.

With a view to the mean particle size, four ranges may be preferred. In a first embodiment, the composition according to the invention comprises pregabaline with a mean particle size of 0.01 to 50 μm, preferably 0.1 to 20 μm, more preferably 1 to less than 10 μm, in particular 2 to 5 μm. The particle size of this embodiment can, for example, be achieved by means of the “spiral mill AS 50” from Hosokawa, an injection and grinding gas pressure of approx. 2 bar being preferred. Within the context of this invention, particles of the first embodiment are referred to as “micronised pregabaline”.

In the second preferred embodiment, the composition according to the invention comprises pregabaline or micronised pregabaline with a mean particle size of above 5 μm.

In the third preferred embodiment, the composition according to the invention comprises pregabaline with a mean particle size of 50 to 250 μm, preferably 80 to 150 μm, more preferably 90 to 130 μm, in particular approx. 120 μm.

In the fourth preferred embodiment, the composition according to the invention comprises pregabaline with a mean particle size of more than 250 μm to 1.3 mm, preferably of 400 μm to 1.0 mm, more preferably 600 μm to 800 μm, in particular approx. 680 μm.

Within the context of this invention, the mean particle size, which is also referred to as D₅₀ value is, in defined as the particle size in the case of which 50% of the particles, based on the volume, are smaller than the D₅₀ value and 50% of the particles, based on the volume, are larger than the D₅₀ value. The determination of the particle size was carried out by means of a “Mastersizer 2000” device from “Malver Instruments”. The determination is preferably carried out according to DIN 13320-1. The dry dispersion unit “Scirocco 2000” was used for this purpose. For the determination of the particle size distribution, the powder to be examined was introduced into the product feed facility and passed through the lense chamber by shaking and reduced pressure and detected. To eliminate agglomerates, a dispersion pressure of 0-4 bar was added, where necessary. The measurement took place according to DIN 13320-1, the Frauenhofer method of measurement or measurement evaluation being used.

In a preferred embodiment, component (b) of the pharmaceutical composition contains alkaline earth phosphates as auxiliary substances. In this respect, magnesium phosphate and calcium phosphate are preferred, calcium phosphate being particularly preferred. Examples of suitable calcium phosphate compounds are tribasic calcium phosphate (Ca₃(PO₄)₂), calcium dihydrogen phosphate monohydrate (Ca(H₂PO₄)₂×1H₂O), calcium hydrogen phosphate dihydrate (CaHPO₄×2H₂O) or calcium hydrogen phosphate anhydrate (CaHPO₄). In particular, calcium hydrogen phosphate dihydrate (CaHPO₄×2H₂O) or calcium hydrogen phosphate anhydrate (CaHPO₄) are used.

In a further preferred embodiment, component (b) of the pharmaceutical composition contains hexites (hexahydric alcohols) and/or pentites (petahydric alcohols). Examples of hexites are sorbitol, mannitol or dulcitol. Preferred examples of hexites are mannitol and dulcitol. A particularly preferred example of a hexite is mannitol. Examples of pentites are arabinitol, adonitol or xylitol.

The composition according to the invention contains, in a further preferred embodiment, a polyacrylate as pharmaceutical auxiliary substance.

Preferably, carboxypolymethylene and/or carboxyvinyl polymers are used as polyacrylate. The polyacrylates marketed under the brand names of “Carbopol®” and “Carbomer®” are preferably also used. The polyacrylates used preferably have a weight average molecular weight of 100,000 to 5 million g/mole, preferably of 500,000 to 2 million g/mole, more preferably of 800,000 to 1 million g/mole.

The subject matter of the invention is consequently also a pharmaceutical composition containing

(a) pregabaline as active principle and

(b) alkaline earth phosphates as pharmaceutical auxiliary substances, in particular calcium hydrogen phosphate dihydrate (CaHPO₄×2H₂O) or calcium hydrogen phosphate anhydrate (CaHPO₄).

The subject matter of the invention is moreover also a pharmaceutical composition containing

(a) pregabaline as active principle and

(b) hexites (hexahydric alcohols) and/or pentites (pentahydric alcohols) as pharmaceutical auxiliary substances.

The subject matter of the invention is also a pharmaceutical composition containing

(a) pregabaline as active principle and

(b) polyacrylate as pharmaceutical auxiliary substance.

Within the context of the invention, the term pharmaceutical composition should preferably understood to mean that a mixture (in particular an intimate mixture) between the active principle (pregabaline) and the auxiliary substance is present. This means that the auxiliary substances used are in direct contact with the active principle. The pharmaceutical composition is thus present in the form of a mixture with contact between the active principle and the auxiliary substance.

Compositions in the case of which pregabaline is encapsulated with a polymer, for example, and the capsules are embedded in a hexite matrix, consequently do not preferably come under the subject matter of the present application since no direct contact exists between pregabaline and hexite.

The term “pharmaceutical composition” should, moreover, be understood to mean that it is a composition which is suitable for administration to a patient, e.g. in the form of tablets or capsules. The term should not be understood to mean that is comprises powders which are not suitable for administration but are used e.g. only for stability tests.

It also need to be noted that the expressions “essentially free from saccharides” and “no further amino acids, apart from pregabaline” relate to the pharmaceutical composition as such. These expressions do not exclude the possibility that tablets, for example, which have been pressed from the pharmaceutical composition according to the invention as such are coated with a saccharide-containing film or are introduced into amino acid-containing capsules.

The pharmaceutical composition is a solid composition. The pharmaceutical composition according to the invention is present in particular in the form of tablets or capsules. Alternatively, the pharmaceutical composition according to the invention can also be present in the form of a dry powder for reconstitution.

The composition according to the invention generally contains 10 to 90% by weight of pregabaline, based on the total weight of the composition. Preferably, the composition according to the invention contains 20 to 80% by weight of pregabaline.

The quantity of pregabaline may be dependent on the form of presentation.

Insofar as it consists of tablets and capsules of up to 80 mg pregabaline, these preferably contain 10 to 50% by weight of pregabaline, more preferably 20 to 30% by weight of pregabaline, in particular 25% by weight of pregabaline, based on the total weight of the pharmaceutical composition.

Insofar as tables or capsules with more than 80 mg pregabaline are involved these preferably contain 40 to 90% by weight of pregabaline, more preferably 60 to 85% by weight of pregabaline, in particular 70 to 80% by weight of pregabaline, based on the total weight of the pharmaceutical composition.

The composition according to the invention moreover generally contains 10 to 90% by weight of pharmaceutical auxiliary substances (b), based on the total weight of the composition. Preferably, the composition according to the invention contains 40 to 80% by weight of pharmaceutical auxiliary substances (b).

It is also preferably to use a mixture of alkaline earth phosphates, on the one hand, and of hexites and/or pentites, on the other hand, as component (b). Preferably, 10 to 90% by weight of alkaline earth phosphates, on the one hand, and 10 to 90% by weight hexites and/or pentites, on the other hand, more preferably, 20 to 80% by weight of alkaline earth phosphates, on the one hand, and 20 to 80% by weight of hexites and/or pentites, on the other hand, are used, even more preferably, 30 to 70% by weight of alkaline earth phosphates, on the one hand, and 30 to 70% by weight of hexites and/or pentites, on the other hand, are used, in particular 40 to 60% by weight of alkaline earth phosphates, on the one hand, and 40 to 60% by weight of hexites and/or pentites, on the other hand, are used.

Preferably, a mixture of calcium phosphate and mannitol is used in particular in the above-mentioned quantitative ratios. With respect to calcium phosphates, the above explanations regarding preferred embodiments are applicable.

The above-mentioned alkaline earth phosphates and the hexites and/or pentites are preferably used as fillers in the composition according to the invention. In addition, the composition according to the invention may also contain binders, lubricants, disintegrating agents and fluxes.

Suitable binders comprise for example povidone, crospovidone, polyvinylpyrrolidone, polyethylene glycol, wax or mixtures thereof. In general, binders can be used in a quantity of 0 to 30% by weight, preferably of 1 to 10% by weight, based on the total weight of the composition.

Suitable lubricants comprise, for example, sodium oleate, sodium stearate, magnesium stearate, sodium benzoate, sodium acetate, sodium chloride, stearic acid, sodium stearyl fumarate, sodium dodecyl sulphate (SDS), talcum or mixtures thereof. In general, lubricants are used in a quantity of 0 to 40% by weight, preferably of 1 to 20% by weight, based on the total weight of the pharmaceutical composition.

In a preferred embodiment, talcum is used as lubricant. Preferably, talcum is used in a quantity of 2 to 25% by weight, in particular 4 to 20% by weight, based on the total weight of the pharmaceutical composition.

In a further preferred embodiment, sodium dodecyl sulphate (SDS) is used as lubricant. Preferably, sodium dodecyl sulphate (SDS) is used in a quantity of 0.1 to 5% by weight, in particular 1 to 2% by weight, based on the total weight of the pharmaceutical composition. It has unexpectedly been found that this embodiment is particularly advantageous with respect to the bioavailability.

In a further preferred embodiment, magnesium stearate is used a lubricant. Preferably, magnesium stearate is used in a quantity of 1 to 10% by weight, in particular 2 to 5% by weight, based on the total weight of the pharmaceutical composition.

It is possible to use, as flux, e.g. aluminium stearate, calcium stearate, magnesium stearate, wax, aluminosilicate, palmitic acid, stearyl alcohol and silicon dioxide. Silicon dioxide, in particular highly dispersed silicon dioxide, is particularly preferred as flux. Suitable fluxes are usually marketed under the brand name of “Aerosil”. Fluxes are usually used in a quantity of 0.1 to 20% by weight, preferably 0.2 to 10, in particular 0.5 to 5% by weight, based on the total weight of the pharmaceutical composition.

Insofar as silicon dioxide, in particular highly dispersed silicon dioxide, is used as flux this is particularly preferably used in a quantity of 0.5 to 2% by weight, based on the total weight of the pharmaceutical composition.

A flux is used in particular if the pharmaceutical composition according to the invention is used for making tablets. The addition of the flux is, moreover, preferred in particular if component (b) contains calcium phosphate. The expression “calcium phosphate” relates in this case to all forms of calcium phosphate described above.

Disintegrating agents comprise for example bentonite, pyrrolidones, povidone, crospovidone, polyvinyl pyrrolidone and mixtures thereof. Preferred disintegrating agents are bentonite, pyrrolidones, povidone, polyvinyl pyrrolidone and mixtures thereof. Particularly preferred disintegrating agents are Kolidon®, Kolidon VA®, Kolidon CL®.

The composition according to the invention can preferably be present as a capsule, tablet, pellet or dry powder for reconstitution. The subject matter of the invention consequently also comprises a capsule, tablet, pellet or dry powder for reconstitution containing the composition according to the invention. Preferred embodiments for tablets are IR tablets, micro-tablets, ER tablets and tablets disintegrating in the mouth.

Insofar as the composition according to the invention is present as dry powder for reconstition, this preferably contains 40 to 60% by weight polyacrylate, in particular approx. 50% by weight polyacrylate, based on the total weight of the composition.

The present invention comprises numerous embodiments. Basically, the present invention comprises also all combinations of preferred embodiments described, e.g. the combination of the preferred pharmaceutical auxiliary substances with the preferred pregabaline particle size and the preferred form of presentation.

In the following, the particularly preferred combinations are to be presented once more:

pregabaline+calcium phosphate;

micronised pregabaline+calcium phosphate;

pregabaline+calcium phosphate+SDS;

micronised pregabaline+calcium phosphate+SDS;

pregabaline+calcium phosphate+flux;

micronised pregabaline+calcium phosphate+flux;

pregabaline+calcium phosphate+SDS+flux;

micronised pregabaline+calcium phosphate+SDS+flux;

pregabaline+calcium phosphate in the form of a tablet;

micronised pregabaline+calcium phosphate in the form of a tablet;

pregabaline+calcium phosphate+SDS in the form of a tablet;

micronised pregabaline+calcium phosphate+SDS in the form of a tablet;

pregabaline+calcium phosphate+flux in the form of a tablet;

micronised pregabaline+calcium phosphate+flux in the form of a tablet;

pregabaline+calcium phosphate+SDS+flux in the form of a tablet;

micronised pregabaline+calcium phosphate+SDS+flux in the form of a tablet;

pregabaline+calcium phosphate in the form of a capsule;

micronised pregabaline+calcium phosphate in the form of a capsule;

pregabaline+calcium phosphate+SDS in the form of a capsule;

micronised pregabaline+calcium phosphate+SDS in the form of a capsule;

pregabaline+calcium phosphate+flux in the form of a capsule;

micronised pregabaline+calcium phosphate+flux in the form of a capsule;

pregabaline+calcium phosphate+SDS+flux in the form of a capsule;

micronised pregabaline+calcium phosphate+SDS+flux in the form of a capsule;

pregabaline+mannitol;

micronised pregabaline+mannitol;

pregabaline+mannitol+SDS;

micronised pregabaline+mannitol+SDS;

pregabaline+mannitol+flux;

micronised pregabaline+mannitol+flux;

pregabaline+mannitol+SDS+flux;

micronised pregabaline+mannitol+SDS+flux;

pregabaline+mannitol in the form of a tablet;

micronised pregabaline+mannitol in the form of a tablet;

pregabaline+mannitol+SDS in the form of a tablet;

micronised pregabaline+mannitol+SDS in the form of a tablet;

pregabaline+mannitol+flux in the form of a tablet;

micronised pregabaline+mannitol+flux in the form of a tablet;

pregabaline+mannitol+SDS+flux in the form of a tablet;

micronised pregabaline+mannitol+SDS+flux in the form of a tablet;

pregabaline+mannitol in the form of a capsule;

micronised pregabaline+mannitol in the form of a capsule;

pregabaline+mannitol+SDS in the form of a capsule;

micronised pregabaline+mannitol+SDS in the form of a capsule;

pregabaline+mannitol+flux in the form of a capsule;

micronised pregabaline+mannitol+flux in the form of a capsule;

pregabaline+mannitol+SDS+flux in the form of a capsule;

micronised pregabaline+mannitol+SDS+flux in the form of a capsule;

pregabaline with a mean particle size >5 μm+calcium phosphate;

micronised pregabaline with a mean particle size >5 μm+calcium phosphate;

pregabaline with a mean particle size >5 μm+calcium phosphate+SDS;

micronised pregabaline with a mean particle size >5 μm+calcium phosphate+SDS;

pregabaline with a mean particle size >5 μm+calcium phosphate+flux;

micronised pregabaline with a mean particle size >5 μm+calcium phosphate+flux;

pregabaline with a mean particle size >5 μm+calcium phosphate+SDS+flux;

micronised pregabaline with a mean particle size >5 μm+calcium phosphate+SDS+flux;

pregabaline with a mean particle size >5 μm+calcium phosphate in the form of a tablet;

micronised pregabaline with a mean particle size >5 μm+calcium phosphate in the form of a tablet;

pregabaline with a mean particle size >5 μm+calcium phosphate+SDS in the form of a tablet;

micronised pregabaline with a mean particle size >5 μm+calcium phosphate+SDS in the form of a tablet;

pregabaline with a mean particle size >5 μm+calcium phosphate+flux in the form of a tablet;

micronised pregabaline with a mean particle size >5 μm+calcium phosphate+flux in the form of a tablet;

pregabaline with a mean particle size >5 μm+calcium phosphate+SDS+flux in the form of a tablet;

micronised pregabaline with a mean particle size >5 μm+calcium phosphate+SDS+flux in the form of a tablet;

pregabaline with a mean particle size >5 μm+calcium phosphate in the form of a capsule;

micronised pregabaline with a mean particle size >5 μm+calcium phosphate in the form of a capsule;

pregabaline with a mean particle size >5 μm+calcium phosphate+SDS in the form of a capsule;

micronised pregabaline with a mean particle size >5 μm+calcium phosphate+SDS in the form of a capsule;

pregabaline with a mean particle size >5 μm+calcium phosphate+flux in the form of a capsule;

micronised pregabaline with a mean particle size >5 μm+calcium phosphate+flux in the form of a capsule;

pregabaline with a mean particle size >5 μm+calcium phosphate+SDS+flux in the form of a capsule;

micronised pregabaline with a mean particle size >5 μm+calcium phosphate+SDS+flux in the form of a capsule;

pregabaline with a mean particle size >5 μm+mannitol;

micronised pregabaline with a mean particle size >5 μm+mannitol;

pregabaline with a mean particle size >5 μm+mannitol+SDS;

micronised pregabaline with a mean particle size >5 μm+mannitol+SDS;

pregabaline with a mean particle size >5 μm+mannitol+flux;

micronised pregabaline with a mean particle size >5 μm+mannitol+flux;

pregabaline with a mean particle size >5 μm+mannitol+SDS+flux;

micronised pregabaline with a mean particle size >5 μm+mannitol+SDS+flux;

pregabaline with a mean particle size >5 μm+mannitol in the form of a tablet;

micronised pregabaline with a mean particle size >5 μm+mannitol in the form of a tablet;

pregabaline with a mean particle size >5 μm+mannitol+SDS in the form of a tablet;

micronised pregabaline with a mean particle size >5 μm+mannitol+SDS in the form of a tablet;

pregabaline with a mean particle size >5 μm+mannitol+flux in the form of a tablet;

micronised pregabaline with a mean particle size >5 μm+mannitol+flux in the form of a tablet;

pregabaline with a mean particle size >5 μm+mannitol+SDS+flux in the form of a tablet;

micronised pregabaline+mannitol+SDS+flux in the form of a tablet;

pregabaline with a mean particle size >5 μm+mannitol in the form of a capsule;

micronised pregabaline with a mean particle size >5 μm+mannitol in the form of a capsule;

pregabaline with a mean particle size >5 μm+mannitol+SDS in the form of a capsule;

micronised pregabaline with a mean particle size >5 μm+mannitol+SDS in the form of a capsule;

pregabaline with a mean particle size >5 μm+mannitol+flux in the form of a capsule;

micronised pregabaline with a mean particle size >5 μm+mannitol+flux in the form of a capsule;

pregabaline with a mean particle size >5 μm+mannitol+SDS+flux in the form of a capsule;

micronised pregabaline with a mean particle size >5 μm+mannitol+SDS+flux in the form of a capsule.

The subject matter of the present invention consequently also consists of the use of an alkaline earth phosphate for the production of a pharmaceutical pregabaline formulation.

In addition, the subject matter of the invention consists of the use of a pentite and/or hexite for the production of a pharmaceutical pregabaline formulation.

Finally, the subject matter of the invention consists of the use of a polyacrylate for the production of a pharmaceutical pregabaline formulation, in particular a pregabaline dry powder for reconstitution.

For the applications according to the invention, the above explanations regarding the preferred embodiments concerning the pharmaceutical composition according to the invention are applicable.

The invention is to be illustrated by the following figures and examples.

### FIGURES

FIGS. 1, 3 and 5 represent dissolution profiles.

FIGS. 2, 4 and 6 represent results of XRPD analyses.

### EXAMPLES

### Example 1

Pregabaline capsules containing calcium phosphate anhydrate. Mean particle size >5 μm:

| No. | Substance | Function | [mg] |
|---|---|---|---|
| 1 | Pregabaline | API | 25 |
| 2 | Calcium | filler | 55 |
| | hydrogen | | |
| | phosphate | | |
| | anhydrate | | |
| 3 | talcum | lubricant | 20 |
| 4 | total | | 100.00 |

Firstly, pregabaline, being the active ingredient, and calcium phosphate anhydrate were weighed in and mixed.

Subsequently, the addition of the lubricant and renewed mixing were carried out. After sieving through a 0.5 mm sieve the mixture was mixed for additional 10 minutes.

Subsequently, the composition was introduced into capsules (capsule size 4).

Mixing device: Turbula T10B, 30 rpm

Target weight (API): 25.00 mg/capsule

Target weight(per capsule): 100.00 mg

API Potency: 99.64%

Batch size: 300 capsules

The capsules and the reference-product Lyrica® 25 mg were stored at 60° C. for 4 weeks (stress stability test) and at 40° C. at a relative atmospheric humidity of 75% for 12 weeks.

Two different lots having two different chemical grades were stored: Lot ALV07081AL5, where Calcium-hydrogenphosphat anhydrate was used and lot ALV07129AL6, where Calcium-hydrogenphosphat Emcompress Anhydrous was used.

TABLE 1
Lot ALV07081AL5 storage 60° C. for 4 weeks

| | | | | reference-product | |
|---|---|---|---|---|---|
| analysis | | initially | 4 weeks | initially | 4 weeks |
| water content (KF) [%] | | 0.10 | 0.00 | 3.60 | 3.40 |
| purity | Max Single | n/d | 0.20 | n/d | 0.68 |
| (HPLC) | Unknown [%] | | | | |
| | Total [%] | n/d | 0.27 | 0.01 | 2.08 |

n/d—not detectable

TABLE 2
Lot ALV07081AL5 storage at 40° C. at a relative
atmospheric humidity of 75% for 12 weeks.

| | | | | reference-product | |
|---|---|---|---|---|---|
| analysis | | initially | 12 weeks | initially | 12 weeks |
| water content (KF) [%] | | 0.1 | 0.1 | 3.60 | 3.50 |
| purity | Max Single | n/d | 0.25 | 0.69 | 0.35 |
| (HPLC) | Unknown [%] | | | | |
| | Total [%] | n/d | 0.30 | 0.01 | 1.26 |

n/d—not detectable

Water content, pregabalin content and optical purity were stable during storage.

TABLE 3
Lot ALV07129AL6 storage at 40° C. at a relative
atmospheric humidity of 75% for 12 weeks.

| | | | | reference-product | |
|---|---|---|---|---|---|
| analysis | | initially | 12 weeks | initially | 12 weeks |
| water content (KF) [%] | | | | | |
| dissolution | 5 min [%] | 75.1 | 81.2 | 61.5 | 45.4 |
| | mean value | | | | |
| | 15 min [%] | 93.8 | 93.7 | 96.1 | 90.1 |
| | mean value | | | | |
| purity | Max Single | 0.20 | 0.22 | 0.69 | 0.35 |
| (HPLC) | Unknown [%] | | | | |
| | Total [%] | 0.65 | 0.37 | 0.01 | 1.26 |

n/d—not detectable

Water content, pregabalin content and optical purity were stable during storage.

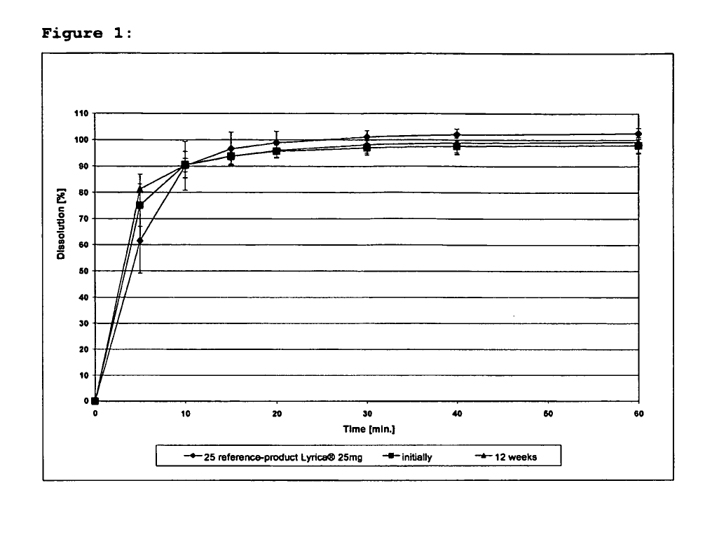

FIG. 1 represents the dissolution profile of lot ALV071291AL6 initially and after storage at 40° C. at a relative atmospheric humidity of 75% for 12 weeks.

Conditions: 500 mL 0.1 N HCl; pH 1.1; 37° C.; 50 rpm paddle (USP App. II)

No significant change of the dissolution profile could be observed after storage.

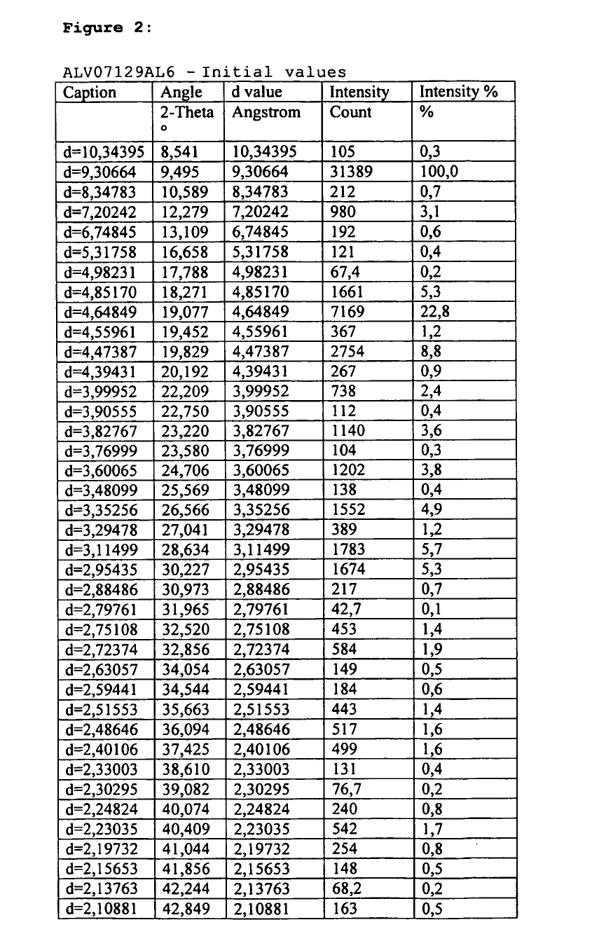

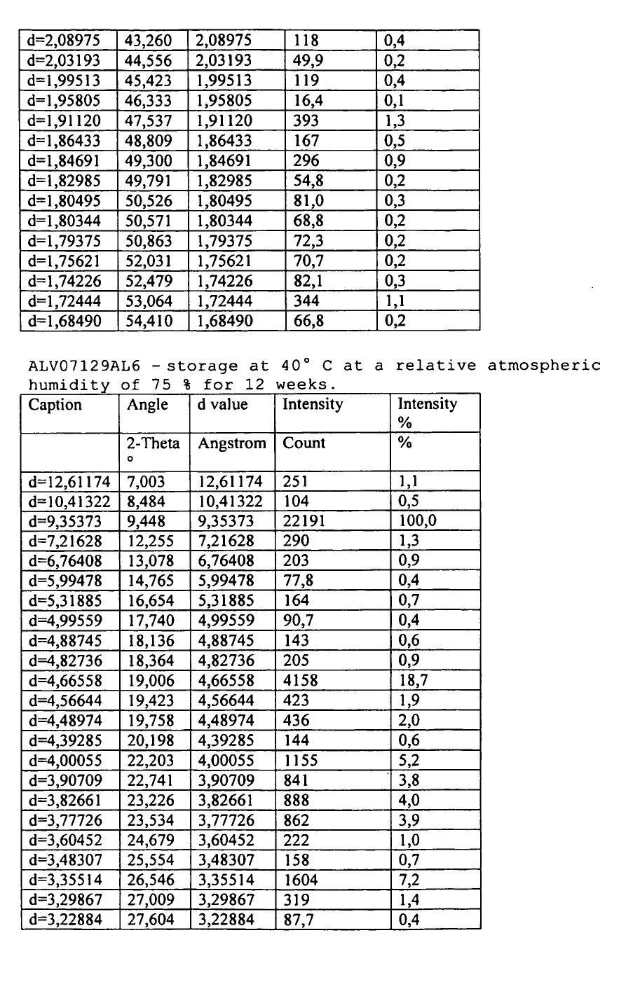

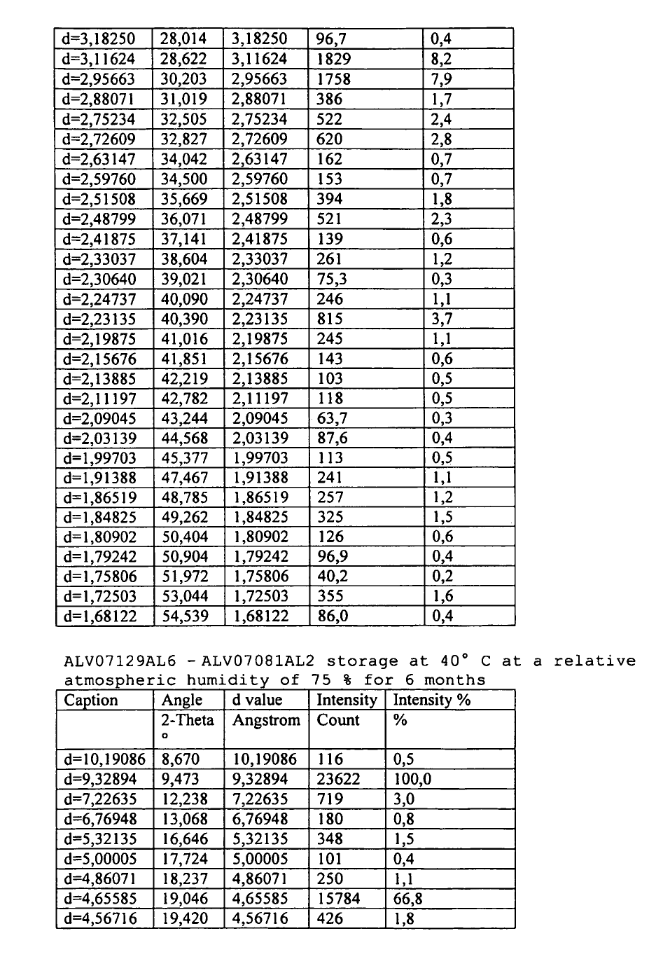

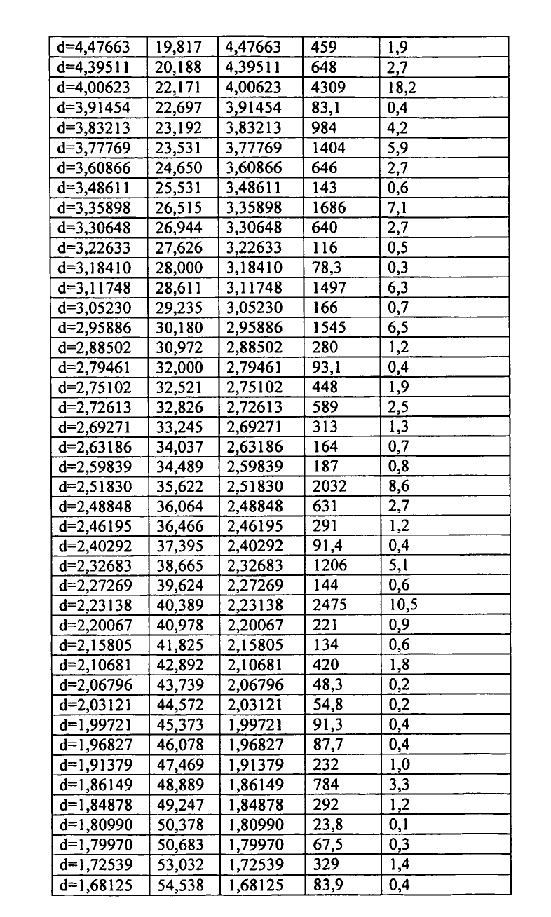

Influence of storage on crystal structure was analysed by XRPD. The results are represented in the table submitted herewith as FIG. 2 (XRPD of lot ALV07129AL6).

### Example 1a

Capsules are produced as in example 1, SDS (2% by weight) being added to the mixture.

### Example 1b

### Micronised

Capsules are produced as in example 1, pregabaline with an mean particle size of 5 μm being used.

Calciumhydrogenphosphat-Anhydrat was used (ALV07081AL6).

TABLE 4
Lot ALV07081AL6 storage at 60° C. for 4 weeks

| | | | | reference-product | |
|---|---|---|---|---|---|
| analysis | | initially | 12 weeks | initially | 12 weeks |
| water content (KF) [%] | | 0.1 | 0.00 | 3.60 | 3.40 |
| purity | Max Single | n/d | 0.19 | n/d | 0.68 |
| (HPLC) | Unknown [%] | | | | |
| | Total [%] | >0.01 | 0.47 | 0.01 | 2.08 |

n/d—not detectable

Water content, pregabalin content and optical purity were stable during storage.

TABLE 5
Lot ALV07081AL6 storage at 40° C. at a relative
atmospheric humidity of 75% for 12 weeks.

| | | | | reference-product | |
|---|---|---|---|---|---|
| analysis | | initially | 12 weeks | initially | 12 weeks |
| water content (KF) [%] | | 0.1 | 0.1 | 3.60 | 3.50 |
| purity | Max Single | n/d | 0.25 | 0.69 | 0.35 |
| (HPLC) | Unknown [%] | | | | |
| | Total [%] | <0.01 | 0.49 | 0.01 | 1.26 |

n/d—not detectable

Water content, pregabalin content and optical purity were stable during storage.

### Example 1c

### Flux

Capsules are produced as in example 1, with additionally 1% by weight of highly dispersed silicon dioxide being added to the mixture.

### Example 2

Pregabaline capsules containing mannitol; mean particle size >5 μm:

In line with example 1, the following components are processed to a capsule:

| No. | Substance | Function | [mg] |
|---|---|---|---|
| 1 | Pregabaline | API | 25 |
| 2 | mannitol | filler | 55 |
| 3 | talcum | lubricant | 20 |
| 4 | total | | 100.00 |

TABLE 6
Lot ALV07081AL1 storage at 60° C. for 4 weeks

| | | | | reference-product | |
|---|---|---|---|---|---|
| analysis | | initially | 12 weeks | initially | 12 weeks |
| water content (KF) [%] | | 0.10 | 0.00 | 3.60 | 3.40 |
| purity | Max Single | n/d | 0.21 | n/d | 0.68 |
| (HPLC) | Unknown [%] | | | | |
| | Total [%] | n/d | 0.24 | 0.01 | 2.08 |

n/d—not detectable

Water content, pregabalin content and optical purity were stable during storage.

TABLE 7
Lot ALV07081AL1 storage at 40° C. at a relative
atmospheric humidity of 75% for 12 weeks.

| | | | | reference-product | |
|---|---|---|---|---|---|
| analysis | | initially | 12 weeks | initially | 12 weeks |
| water content (KF) [%] | | 0.1 | 0.1 | 3.60 | 3.50 |
| purity | Max Single | n/d | 0.26 | 0.69 | 0.35 |
| (HPLC) | Unknown [%] | | | | |
| | Total [%] | n/d | 0.27 | 0.01 | 1.26 |

n/d—not detectable

Water content, pregabalin content and optical purity were stable during storage.

For the production of lot ALV07129AL3 Mannitol DC400 was used.

TABLE 8
Lot ALV07129AL3 storage at 40° C. at a relative
atmospheric humidity of 75% for 12 weeks.

| | | | | reference-product | |
|---|---|---|---|---|---|
| analysis | | initially | 12 weeks | initially | 12 weeks |
| water content (KF) [%] | | 0.1 | 0.1 | 3.60 | 3.50 |
| dissolution | 5 min [%] | 81.3 | 63.8 | 61.5 | 45.4 |
| | mean value | | | | |
| | 15 min [%] | 96.0 | 95.8 | 96.1 | 90.1 |
| | mean value | | | | |
| purity | Max Single | 0.21 | 0.28 | 0.69 | 0.35 |
| (HPLC) | Unknown [%] | | | | |
| | Total [%] | 0.52 | 0.37 | 0.01 | 1.26 |

n/d—not detectable

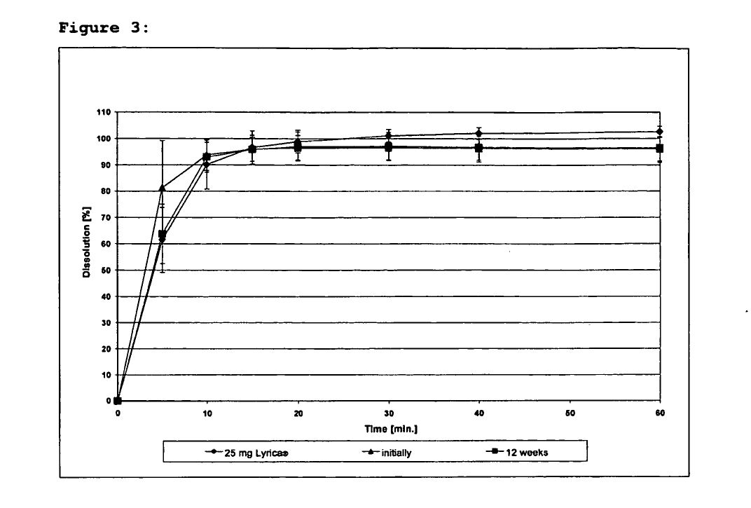

FIG. 3 represents the dissolution profile of lot ALV07129AL3, initially and after storage at 40° C. at a relative atmospheric humidity of 75% for 12 weeks.

Conditions: 500 mL 0.1 N HCl; pH 1.1; 37° C.; 50 rpm paddle (USP App. II)

No significant change of the dissolution profile could be observed.

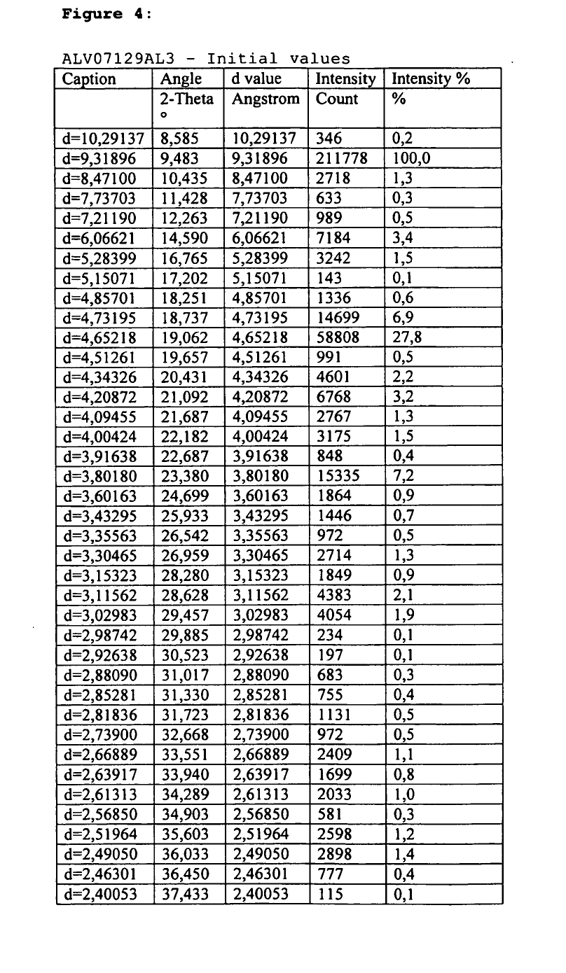

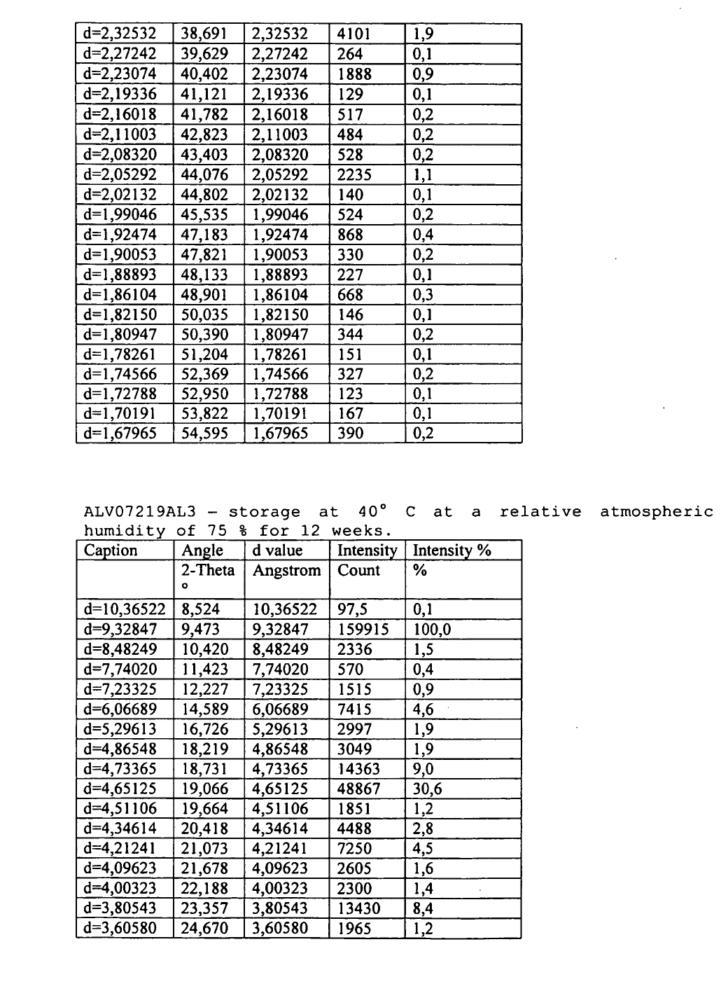

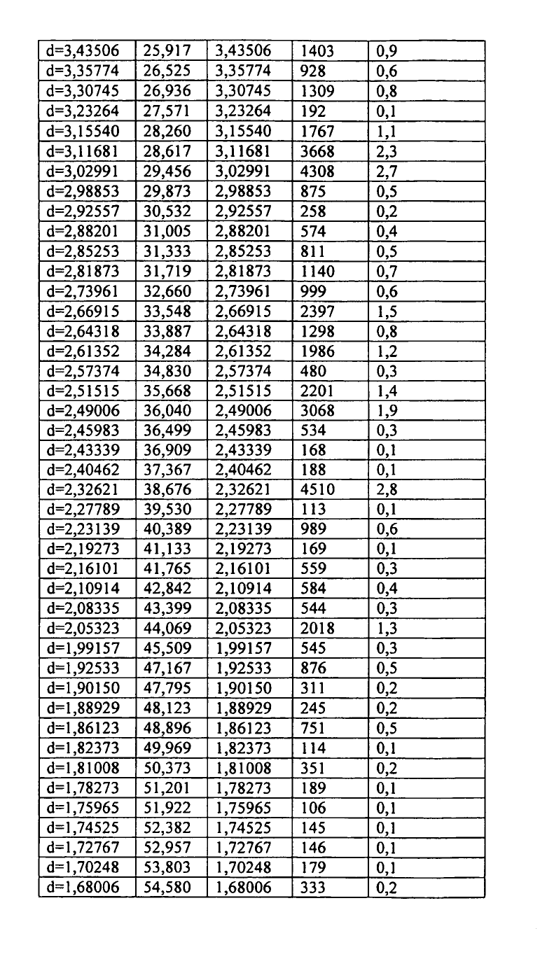

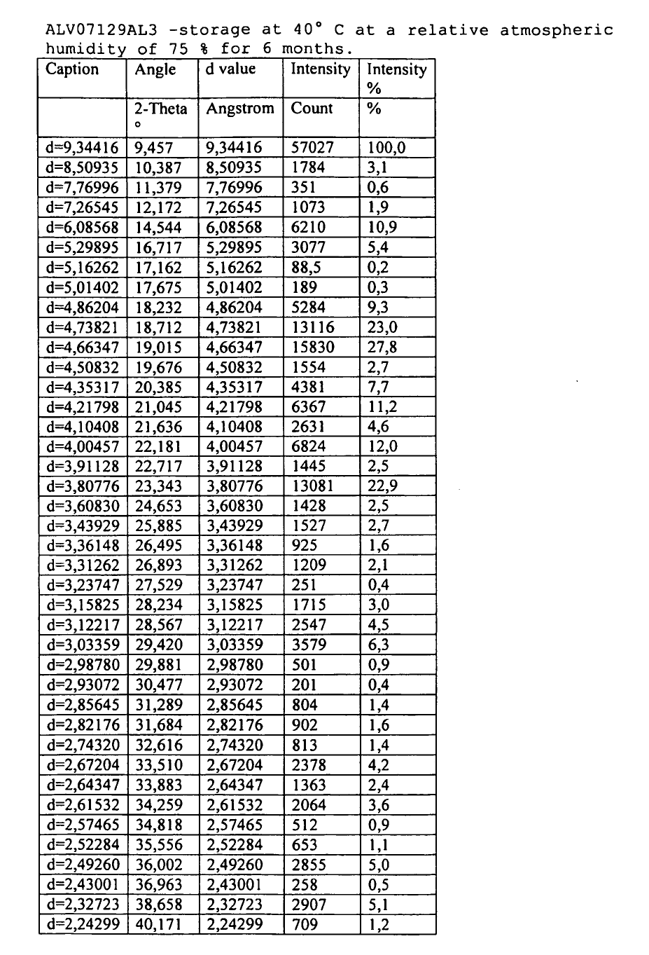

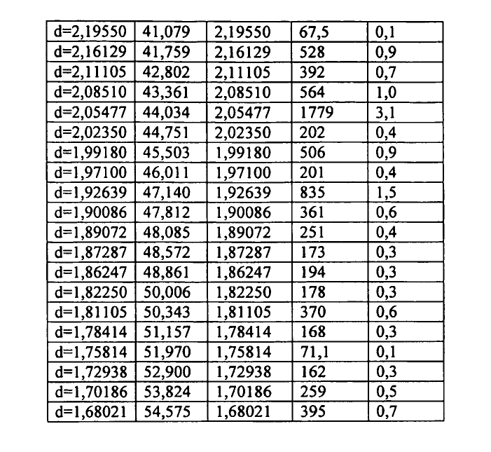

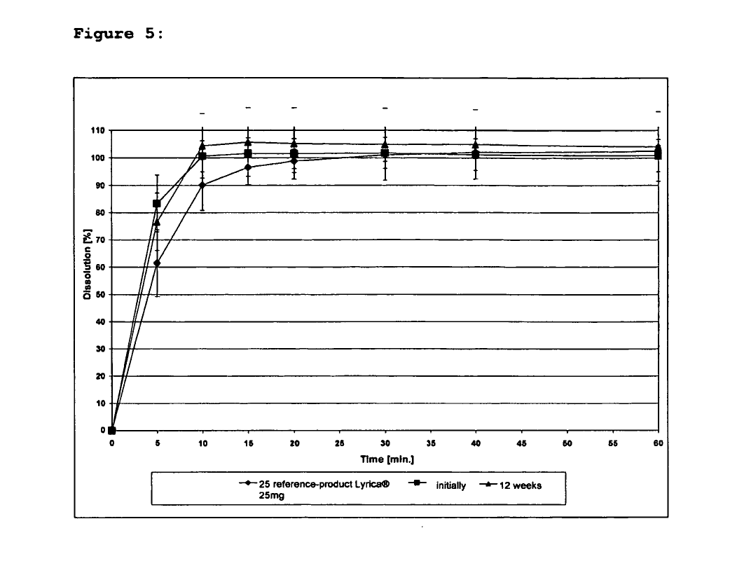

Influence of storage on crystal structure was analysed by XRPD. The results are represented in the table according to FIG. 4 (XRPD of lot ALV07129AL3).

### Example 2a

Capsules are produced as in example 2, SDS (2% by weight) being added to the mixture.

### Example 2b

### Micronised:

Capsules are produced as in example 2, pregabaline with an mean particle size of 5 μm being used.

For the production of lot ALV07081AL2 Mannitol SD 200 was used.

TABLE 9
Lot ALV07081AL2 storage at 60° C. for 4 weeks

| | | | | reference-product | |
|---|---|---|---|---|---|
| analysis | | initially | 12 weeks | initially | 12 weeks |
| water content (KF) [%] | | 0.10 | 0.10 | 3.60 | 3.40 |
| purity | Max Single | n/d | 0.20 | n/d | 0.68 |
| (HPLC) | Unknown [%] | | | | |
| | Total [%] | <0.01 | 0.34 | 0.01 | 2.08 |

n/d—not detectable

Water content, pregabalin content and optical purity were stable during storage.

TABLE 10
Lot ALV07081AL2 storage at 40° C. at a relative
atmospheric humidity of 75% for 12 weeks.

| | | | | reference-product | |
|---|---|---|---|---|---|
| analysis | | initially | 12 weeks | initially | 12 weeks |
| water content (KF) [%] | | 0.1 | 0.1 | 3.60 | 3.50 |
| Dissolution | 5 min [%] | 83.4 | 76.7 | 61.5 | 45.4 |
| | mean value | | | | |
| | 15 min [%] | 101.7 | 105.8 | 96.1 | 90.1 |
| | mean value | | | | |
| purity | Max Single | n/d | 0.26 | 0.69 | 0.35 |
| (HPLC) | Unknown [%] | | | | |
| | Total [%] | <0.01 | 0.37 | 0.01 | 1.26 |

n/d—not detectable

FIG. 5 represents the dissolution profile of lot ALV07081AL2, initially and after storage at 40° C. at a relative atmospheric humidity of 75% for 12 weeks.

Conditions: 500 mL 0.1 N HCl; pH 1.1; 37° C.; 50 rpm paddle (USP App. II)

No significant change of the dissolution profile could be observed after storage.

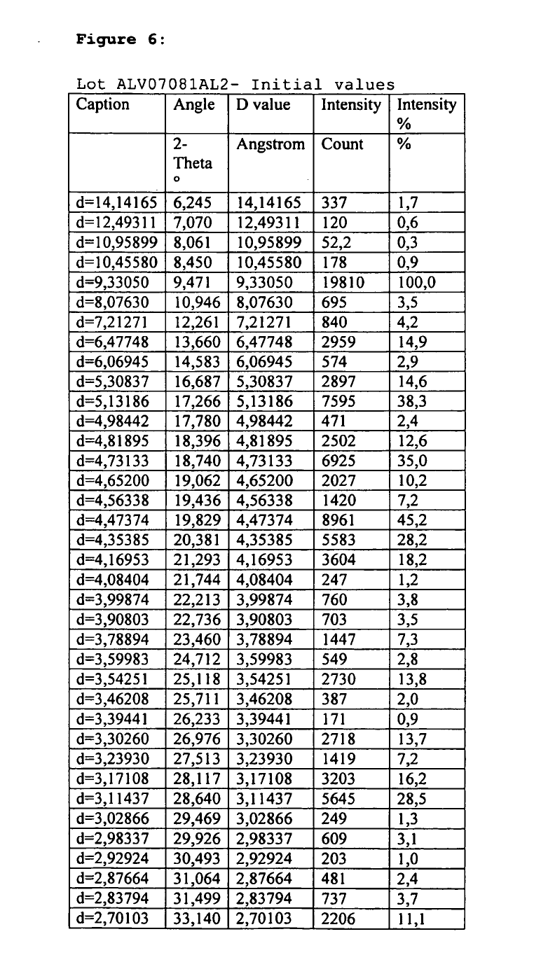

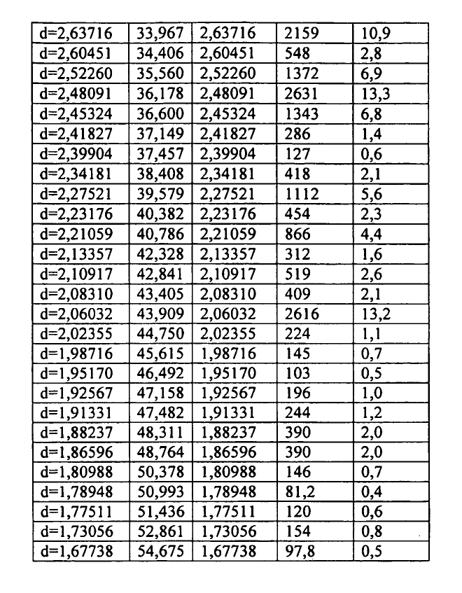

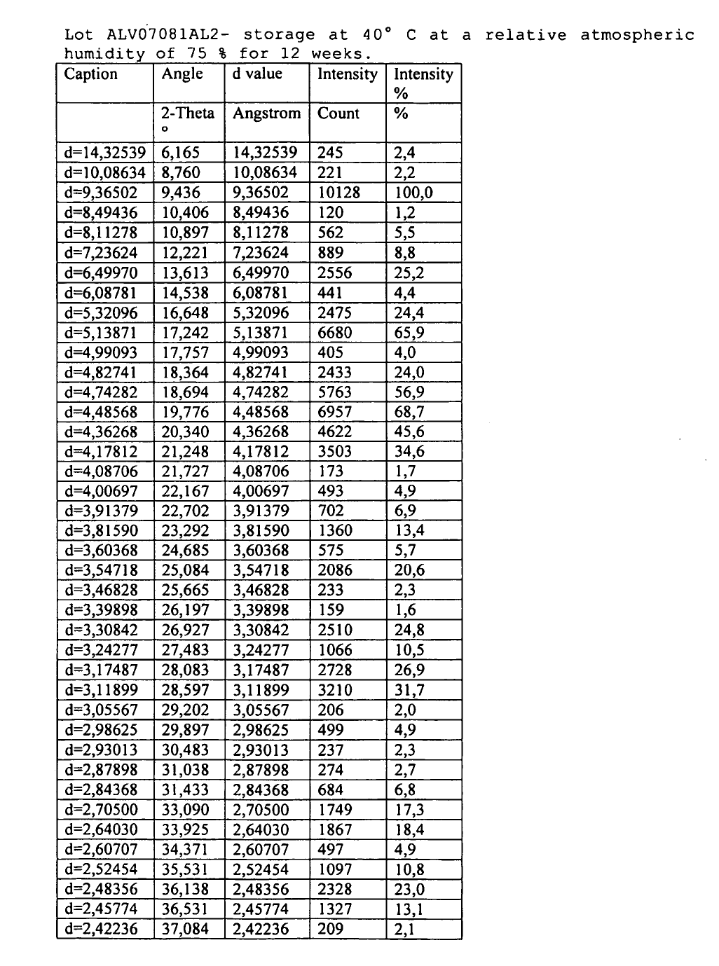

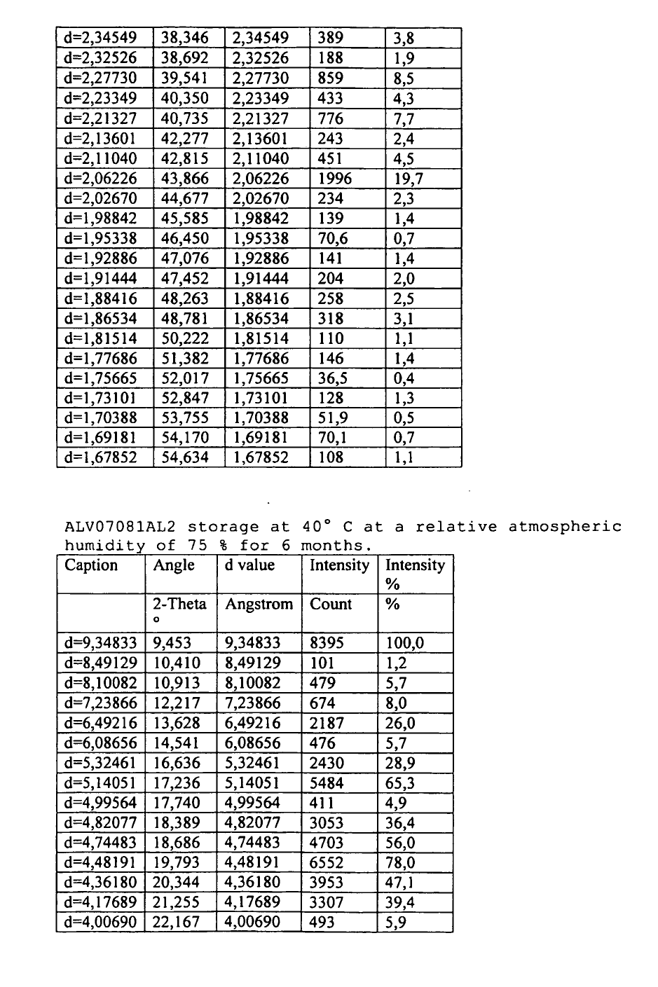

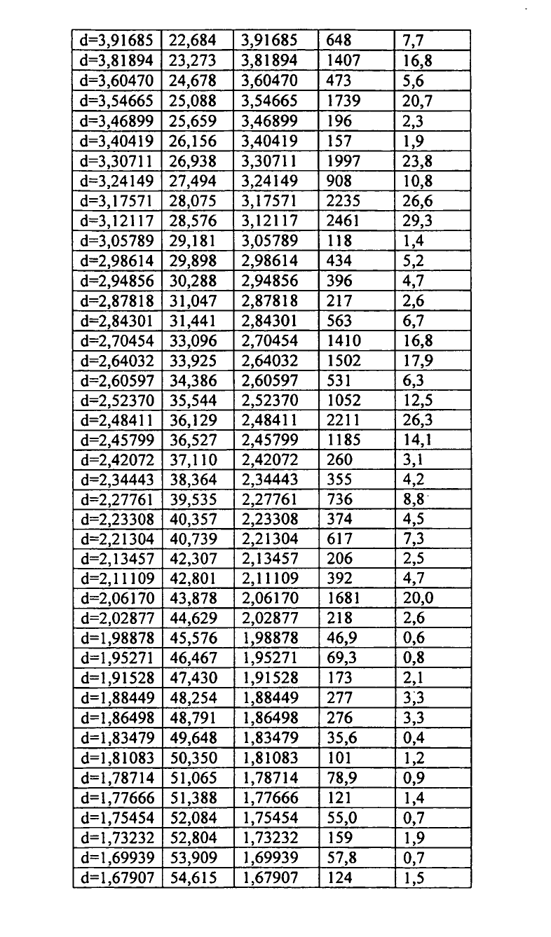

Influence of storage on crystal structure was analysed by XRPD. The results are represented in the table submitted herewith as FIG. 6 (XRPD of lot ALV07081AL2).

For the production of lots ALV07129AL2 Mannitol DC400 was used.

TABLE 11
Lot ALV07129AL2 storage at 60° C. for 4 weeks

| | | | | reference-product | |
|---|---|---|---|---|---|
| analysis | | initially | 12 weeks | initially | 12 weeks |
| water content (KF) [%] | | 0.1 | 0.1 | 3.60 | 3.50 |
| purity | Max Single | 0.22 | 0.27 | 0.69 | 0.35 |
| (HPLC) | Unknown [%] | | | | |
| | Total [%] | 0.61 | 0.37 | 0.01 | 1.26 |

n/d—not detectable

Water content, pregabalin content and optical purity were stable during storage.

### Example 3

### Rapidly Releasing Tables (IRT) Containing Calcium Phosphate Anhydrate:

| No. | Substance | Function | [mg] |
|---|---|---|---|
| 1 | Pregabaline | API | 50 |
| 2 | calcium | filler | 100 |
| | hydrogen | | |
| | phosphate | | |
| | anhydrate | | |
| 3 | Kollidon CL | disintegrating | 10 |
| | | agent | |
| 4 | talcum | lubricant | 35 |
| 5 | magnesium | lubricant | 5 |
| | stearate | | |
| 6 | total | | 200.00 |

Active principle, Kollidon CL and calcium hydrogen phosphate anhydrate were weighed in and mixed. Lubricant was added and mixed again. Subsequently, the tablets were pressed.

### Example 3a

Tablets are produced as in example 3, SDS (2% by weight) being added to the mixture.

### Example 3b

### Micronised

Tablets are produced as in example 3, pregabaline with an mean particle size of 5 μm being used.

### Example 3c

### Flux

Tablets are produced as in example 3, 1% by weight of highly dispersed silicon dioxide being additionally added to the mixture.

### Example 4

Rapidly releasing tablets (IRT) containing mannitol:

As described in example 3, the following components are processed into a tablet:

| No. | Substance | Function | [mg] |
|---|---|---|---|
| 1 | Pregabaline | API | 50 |
| 2 | mannitol | filler | 100 |
| 3 | Kollidon CL | disintegrating | 10 |
| | | agent | |
| 4 | talcum | lubricant | 35 |
| 5 | magnesium | lubricant | 5 |
| | stearate | | |
| 6 | total | | 200.00 |

### Example 5

### Delayed Release Tablets (Matrix Tablet—ERT)

| No. | Substance | Function | [mg] |
|---|---|---|---|
| 1 | Pregabaline | API | 50 |
| 2 | Calcium | filler | 140 |
| | hydrogen | | |
| | phosphate × 2 | | |
| | H₂O | | |
| 3 | talcum | lubricant | 8 |
| 4 | magnesium | lubricant | 5 |
| | stearate | | |
| 5 | total | | 203.00 |

Active principle and calcium hydrogen phosphate×2H₂O were weighed in and mixed. Lubricant was added and mixed again. Subsequently, the mixture was pressed to form tablets.

### Example 5a

Tablets are produced as in example 5, SDS (2% by weight) being added to the mixture.

### Example 5b

### Flux

Delayed release tablets are produced as in example 5, 1% by weight of highly dispersed silicon dioxide being additionally added to the mixture.

### Example 6

### Delayed Release Tablets (Matrix Tablet—ERT)

As described in example 5, the following formulation was processed into tablets:

| No. | Substance | Function | [mg] |
|---|---|---|---|
| 1 | Pregabaline | API | 50 |
| 2 | Calcium | filler | 140 |
| | hydrogen | | |
| | phosphate | | |
| | anhydrate | | |
| 3 | talcum | lubricant | 8 |
| 4 | magnesium | lubricant | 5 |
| | stearate | | |
| 5 | total | | 203.00 |

### Example 6a

### Flux

Delayed release tablets are produced as in example 6, 1% by weight of highly dispersed silicon dioxide being additionally added to the mixture.

### Example 7

Tablets disintegrating in the mouth (ODT):

| No. | Substance | Function | [mg] |
|---|---|---|---|
| 1 | Pregabaline | API | 50 |
| 2 | calcium | filler | 35 |
| | silicate | | |
| 3 | mannitol | filler | 96 |
| 4 | Kollidon CL | disintegrating | 16 |
| | | agent | |
| 5 | magnesium | lubricant | 3 |
| | stearate | | |
| 6 | total | | 200.00 |

Active principle, calcium silicate, mannitol and Kollidon CL were weighed in and mixed. Lubricant was added and mixed again. Subsequently, the mixture was pressed to form tablets.

### Example 7a

Tablets are produced as in example 5, SDS (2% by weight) being added to the mixture.

### Example 8

### Film-Coated Tablets

Film-coated tablets were obtained by film-coating the tablet cores according to examples 3 to 6 with povidones.

### Example 9

Tablets resistant to gastric juice:

Tablets resistant to gastric juice can be achieved using the tablet cores from the exemplary formulations 3 to 6 by coating with Eudragit®L brands.

### Example 10

Redardative tablets:

Redardative tablets can be achieved using the tablet cores from the exemplary formulations 3 to 6 by coating with Eudragit® brands and Kollicoat® brands.

### Example 11

### Pellets

Pregabaline pellets were produced by producing a suspension with pregabaline and Kollidon® VA (vinylpyrrolidone/vinyl acetate copolymer) and spraying the suspension onto mannitol pellets.

### Example 12

### Granules:

Pregabaline granules were produced by granulating pregabaline, mannitol and Kollidon®VA 64.

### Example 13

Dry powder for reconstitution:

| No. | Substance | Function | [mg] |
|---|---|---|---|
| 1 | pregabaline | API | 50 |
| 2 | Carbopol ® | filler | 50 |
| 3 | total | | 100.00 |

Active principle and Carbopol ® were mixed. Subsequently, filling into vials was carried out.

### Example 13a

A dry powder for reconstitution is produced according to example 13, SDS (2% by weight) being added to the mixture.

### Example 13b

### Micronised

A dry powder for reconstitution is produced as in example 13, pregabaline with a mean particle size of 5 μm being used.

1. Solid pharmaceutical composition containing:
(a) pregabaline as active principle and
(b) one or several pharmaceutical auxiliary agents,
the composition being essentially free from saccharides and comprising no further amino acids, apart from pregabaline.

2. Pharmaceutical composition according to claim 1 characterised in that pregabaline particles with a mean particle diameter of less than 250 μm are used as component (a).

3. Pharmaceutical composition according to claim 1 or 2 characterised in that component (b) contains an alkaline earth phosphate, preferably calcium phosphate.

4. Pharmaceutical composition according to claim 3 characterised in that the calcium phosphate is a calcium hydrogen phosphate dehydrate (CaHPO₄×2H₂O) or a calcium hydrogen phosphate anhydrate (CaHPO₄).

5. Pharmaceutical composition according to one of claims 1 to 4 characterised in that component (b) contains pentite and/or hexite.

6. Pharmaceutical composition according to claim 5 characterised in that the hexite is mannitol.

7. Pharmaceutical composition according to one of claims 3 to 6 characterised in that the quantity of alkaline earth phosphate and pentite and/or hexite is 30 to 90% by weight, preferably 40 to 80% by weight, based on the total weight of the pharmaceutical composition.

8. Pharmaceutical composition according to one of claims 1 to 7 characterised in that component (b) contains a polyacrylate.

9. Pharmaceutical composition according to one of claims 1 to 8 characterised in that component (b) contains a surface-active substance, preferably SDS.

10. Tablet, capsule or dry powder for reconstitution containing a pharmaceutical composition according to one of claims 1 to 9.

11. Use of an alkaline earth phosphate for the production of a pharmaceutical pregabaline formulation.

12. Use of a pentite and/or hexite for the production of a pharmaceutical pregabaline formulation.

13. Use of a polyacrylate for the production of a pharmaceutical pregabaline formulation, in particular a pregabaline dry powder for reconstitution.
